(19) United States
(12) Patent Application Publication
(10) Pub. No.: US 2016/0093416 A1
(43) Pub. Date: Mar. 31, 2016

(54) ENDOSCOPE WOVEN CABLE AND ENDOSCOPE CABLE

(71) Applicant: Hitachi Metals, Ltd., Tokyo (JP)

(72) Inventors: Detian HUANG, Hitachi (JP); Takanobu WATANABE, Hitachi (JP); Norihiro NISHIURA, Hitachi (JP)

(21) Appl. No.: 14/847,662

(22) Filed: Sep. 8, 2015

(30) Foreign Application Priority Data
Sep. 29, 2014 (JP) 2014-198617

(51) Int. Cl. H01B 7/16; H01B 9/00; A61B 1/00

(52) U.S. Cl. CPC H01B 7/16; A61B 1/00114; H01B 9/005

(57) ABSTRACT

An endoscope woven cable includes plural cables arranged in parallel, and a filament woven through the plurality of cables in an alignment direction thereof. The plural cables include at least one rigidity-imparting wire. The rigidity-imparting wire may be arranged at both ends and in a middle of the plurality of cables in the alignment direction thereof or symmetrically positioned about a center of the plurality of cables in the alignment direction thereof. An outer diameter of the rigidity-imparting wire may be not greater than that of a rest of the plural cables. The rigidity-imparting wire may include a bare stainless steel wire or a bare steel wire.

The present application is based on Japanese patent application No. 2014-198617 filed on Sep. 29, 2014, the entire contents of which are incorporated herein by reference.

### BACKGROUND OF THE INVENTION

1. Field of the Invention

The invention relates to an endoscope woven cable used for an endoscopic device with a solid-state image sensor such as a CCD (charge coupled device) image sensor or a

CMOS (complementary metal oxide semiconductor) image sensor, and an endoscope cable composed partly of the endoscope woven cable.

2. Description of the Related Art

The solid-state image sensor is used for medical application such as endoscopy or for industrial application such as assembly of microscopic industrial products, and has an endoscope cable in which a multi-core cable is arranged in a flexible hollow cylindrical endoscopic tube. The multi-core cable functions to electrically and/or optically connect an endoscopic device main body with the solid-state image sensor, supply electric power from the endoscopic device main body to the solid-state image sensor, and transmit electrical and/or optical signals between the endoscopic device main body and the solid-state image sensor.

It is proposed that the multi-core cable may be composed of a woven cable formed by weaving a filament through plural parallel cables along an alignment direction of the cables (see e.g., JP-A-2013-062065).

Since the woven cable is flexible and foldable, by using for the multi-core cable, the multi-core cable can be installed in a very small space inside an endoscopic tube while avoiding the physical contact of the multi-core cable with other members arranged inside the endoscopic tube. This helps decrease the diameter of the endoscope cable.

### SUMMARY OF THE INVENTION

In the medical application such as endoscopy, where the endoscope cable is inserted into a patient's body through the mouth etc., the patient may feel uncomfortable with the endoscopy etc. Thus, in order to reduce the uncomfortable feeling of the patient as much as possible, it is desired to further decrease the diameter of the endoscope cable.

In the industrial application such as assembly of microscopic industrial products, where microscopic industrial products have been further miniaturized in recent years, it is necessary to wire the endoscope cable in narrower space. Thus, it is also desired to further decrease the diameter of the endoscope cable in the same manner as the medical application.

In order to further decease the diameter of the endoscope cable, it may be suggested to decrease the diameter of the plural component cables composing the woven cable. However, decreasing the diameter of the cable may increase the flexibility of the woven cable and lower the rigidity of the woven cable, causing poor maneuverability and poor handling properties when the woven cable is inserted into the endoscopic tube. Thus, it may be difficult to appropriately arrange the woven cable inside the endoscopic tube.

Especially in the industrial application such as underground exploration, it is necessary to insert woven cables into a very long, thin endoscopic tube. Thus, it is desired to decrease the diameter of component cables without lowering the rigidity of the woven cable.

It is an object of the invention to provide an endoscope woven cable that prevents the lowering of the rigidity while allowing the reduction of the diameter of the component cables as well as an endoscope cable composed partly of the endoscope woven cable.

(1) According to one embodiment of the invention, an endoscope woven cable comprises:

a plurality of cables arranged in parallel; and

a filament woven through the plurality of cables in an alignment direction thereof,

wherein the plurality of cables comprise at least one rigidity-imparting wire.

In the above embodiment (1) of the invention, the following modifications and changes can be made.

(i) The rigidity-imparting wire is arranged at both ends and in a middle of the plurality of cables in the alignment direction thereof.

(ii) The rigidity-imparting wire is symmetrically positioned about a center of the plurality of cables in the alignment direction thereof.

(iii) An outer diameter of the rigidity-imparting wire is not greater than that of a rest of the plurality of cables.

(iv) The rigidity-imparting wire comprises a bare stainless steel wire or a bare steel wire.

(2) According to another embodiment of the invention, an endoscope cable comprises the endoscope woven cable according to the embodiment (1), wherein the endoscope woven cable is spirally arranged inside an endoscopic tube.

### Effects of the Invention

According to one embodiment of the invention, an endoscope woven cable can be provided that prevents the lowering of the rigidity while allowing the reduction of the diameter of the component cables as well as an endoscope cable composed partly of the endoscope woven cable.

### BRIEF DESCRIPTION OF THE DRAWINGS

Next, the present invention will be explained in more detail in conjunction with appended drawings, wherein:

FIG. 1 is a cross sectional view showing an endoscope woven cable of the present invention;

FIG. 2 is a cross sectional view showing an endoscope cable of the invention; and

FIG. 3 is a perspective phantom view showing the endoscope cable of the invention.

### DETAILED DESCRIPTION OF THE PREFERRED EMBODIMENTS

A preferred embodiment of the invention will be described below in conjunction with the appended drawings.

Firstly, an endoscope woven cable of the invention will be described.

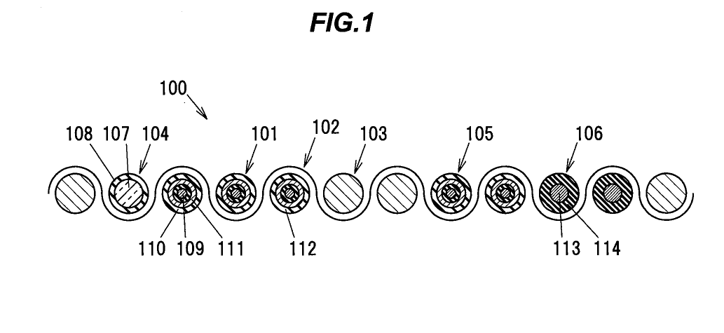

As shown in FIG. 1, an endoscope woven cable 100 in the present embodiment is provided with plural cables 101 arranged in parallel, and a filament 102 woven through the plural cables 101 in an alignment direction of the cables 101.

The endoscope woven cable 100 may additionally have a shielding tape wound therearound, which improves shielding properties of the endoscope woven cable 100.

The plural cables 101 includes at least one rigidity-imparting wire 103. The rest of the cables 101 other than the rigidity-imparting wire(s) 103 are an optical fiber cable(s) 104, a coaxial wire(s) 105 and/or an insulated wire(s) 106 (or may be other types of cables). In other words, the plural cables 101 are composed of the optical fiber cable(s) 104, the coaxial wire(s) 105 and the insulated wire(s) 106, etc., which are arranged in a predetermined order.

The optical fiber cable 104 has an optical fiber 107 formed of silica glass, etc., and a cover layer 108 formed around the optical fiber 107, and is configured to optically connect an endoscopic device main body to a solid-state image sensor and also to transmit optical signals therebetween.

The coaxial wire 105 has an inner conductor 109 formed of a metal wire, an insulation layer 110 formed around the inner conductor 109, an outer conductor 111 formed by spirally winding or braiding metal wires around the insulation layer 110, and a jacket 112 formed around the outer conductor 111, and is configured to electrically connect the endoscopic device main body to the solid-state image sensor and also to transmit electrical signals therebetween.

The metal wires constituting the inner conductor 109 and the outer conductor 111 are formed of copper or aluminum, etc., having excellent electrical conductivity. The metal wire may be a solid wire or a twisted wire, and may have a plated surface.

The insulation layer 110 and the jacket 112 are formed of a fluorine resin such as tetrafluoroethylene perfluoroalkyl vinyl ether copolymer (PFA), tetrafluoroethylene-hexafluoropropylene copolymer (FEP) or ethylene-tetrafluoroethylene copolymer (ETFE), or polyethylene terephthalate (PET).

The insulated wire 106 has a conductor 113 formed of a metal wire and an insulation layer 114 formed around the conductor 113, and is configured to electrically connect the endoscopic device main body to the solid-state image sensor and also to supply electric power from the endoscopic device main body to the solid-state image sensor.

The metal wire constituting the conductor 113 is formed of copper or aluminum, etc., having excellent electrical conductivity. The metal wire may be a solid wire or a twisted wire, and may have a plated surface.

The insulation layer 114 is formed of a fluorine resin such as tetrafluoroethylene perfluoroalkyl vinyl ether copolymer, tetrafluoroethylene-hexafluoropropylene copolymer or ethylene-tetrafluoroethylene copolymer, or polyethylene terephthalate.

Considering that endoscope cables in recent years have an outer diameter of about not less than 3 mm and not more than 5 mm, the plural cables 101 preferably have an outer diameter of about not more than 0.25 mm, specifically, about not less than 0.18 mm and not more than 0.25 mm.

The filament 102 is preferably woven back and forth through the plural cables 101 from one edge to another in a width direction (i.e., in the alignment direction of the plural cables 101) in a zigzag manner throughout the endoscope woven cable 100 from one end to another in a longitudinal direction. In other words, preferably, the filament 102 as a weft is woven through the plural cables 101 (the rigidity-imparting wires 103 and the other cables 101) as warps.

Since the plural cables 101 are bound and gathered at an equal wiring pitch by the filament 102 and are fixed into a flat shape, the width of the endoscope woven cable 100 is theoretically minimized, contributing to downsizing of the endoscope woven cable 100 and leading to reduction in diameter of the endoscope cable.

For convenience, FIG. 1 illustrates as if the filament 102 is cut on both widthwise sides of the endoscope woven cable 100. In practice, however, one filament 102 is continuously woven through the plural cables 101.

In addition, the filament 102 is preferably woven at a weaving density of not less than 10 returns and not more than 20 returns per 1 cm of the endoscope woven cable 100 in the longitudinal direction.

The reason why the filament 102 is woven at a weaving density of not less than 10 returns and not more than 20 returns per 1 cm of the endoscope woven cable 100 in the longitudinal direction is as follows: when the filament 102 is woven at a weaving density of less than 10 returns per 1 cm of the endoscope woven cable 100 in the longitudinal direction, it is not possible to sufficiently bind and fix the plural cables 101. On the other hand, when the filament 102 is woven at a weaving density of more than 20 returns per 1 cm of the endoscope woven cable 100 in the longitudinal direction, flexibility of the endoscope woven cable 100 may be excessively impaired.

Furthermore, the filament 102 woven through the plural cables 101 preferably passes over/under one cable 101 as one unit at the both ends and in the middle of the endoscope woven cable 100 in the width direction.

This configuration allows the filament 102 to reliably bind the plural cables 101, prevents the filament 102 from slipping on the surfaces of the cables 101 and applying non-uniform stress to the cables 101, and also prevents raveling of the filament 102 and resulting release of binding of the plural cables 101.

The middle of the endoscope woven cable 100 in the width direction here is not limited to the exact center of the endoscope woven cable 100 in the width direction and is a concept including the vicinity thereof. Therefore, for example, when the number of the cables 101 constituting the endoscope woven cable 100 is an even number, one of two cables 101 located at the middle of the endoscope woven cable 100 in the width direction can be considered as one unit.

In general, the filament 102 is woven over the entire length of the endoscope woven cable 100 in the longitudinal direction, and the filament 102 woven at the both longitudinal ends of the endoscope woven cable 100 can be removed to facilitate connection to the endoscopic device main body or the solid-state image sensor.

Since the filament 102 woven at the both longitudinal ends of the endoscope woven cable 100 easily ravels by pulling the tip, it is easy to separate the plural cables 101 and the filament 102 only by pulling the tip of the filament 102.

Therefore, special work such as dissolving the filament 102 with a solvent is not necessary for removing the filament 102 woven at the both longitudinal ends of the endoscope woven cable 100, which significantly simplify work of connecting the endoscope woven cable 100 to the endoscopic device main body or the solid-state image sensor and thus allows task of workers or environmental load due to e.g., disposal of the solvent to be significantly reduced.

In addition, the filament 102 is preferably formed of a fiber with a high rate of elongation and a low initial modulus, i.e., a polyurethane elastic fiber of which rate of elongation is not less than 500% and not more than 900%, recovery from 300% elongation is not less than 90% and an initial modulus for 300% elongation is not less than 5 cN/dtex and not more than 30 cN/dtex.

The reason why the rate of elongation is not less than 500% and not more than 900% is as follows: when the rate of elongation is less than 500%, the filament 102 cannot follow bends or twists of the endoscope woven cable 100. On the other hand, when the rate of elongation is more than 900%, the function of the filament 102 to bind and fix the plural cables 101 decreases.

The reason why the recovery from 300% elongation is not less than 90% is as follows: when the recovery from 300% elongation is less than 90%, the filament 102, once elongated with the bends or twists of the endoscope woven cable 100, is less likely to be restored into the original shape even after the bends or twists of the endoscope woven cable 100 are released, and the function of the filament 102 to bind and fix the plural cables 101 decreases.

The reason why the initial modulus for 300% elongation is not less than 5 cN/dtex and not more than 30 cN/dtex is as follows: when the initial modulus for 300% elongation is less than 5 cN/dtex, the filament 102 even woven through the plural cables 101 is not capable of sufficiently binding and fixing the plural cables 101 and it is difficult to manufacture the endoscope woven cable 100 with good appearance. Therefore, a separate post-process is required to reshape the endoscope woven cable 100 and the manufacturing cost thereof thus increases.

Meanwhile, when the initial modulus for 300% elongation is more than 30 cN/dtex, the plural cables 101 are firmly tightened by the filament 102 woven therethrough, which may cause the cables 101 to undulate or to be broken due to the undulating and resulting deterioration in electrical characteristics of the endoscope woven cable 100.

One example of polyurethane elastic fiber satisfying the conditions described above is Roica (trademark) manufactured by Asahi Kasei Corporation. From the viewpoint of improvement in strength and space saving of the endoscope woven cable 100, it is desirable to use a monofilament elastic fiber.

When using the filament 102 formed of a polyurethane elastic fiber which satisfies the conditions described above, it is possible to weave the very fine filament 102 through the plural cables 101 while greatly stretching the filament 102.

For example, it is possible to weave the filament 102 of not less than 17 dtex and not more than 45 dtex at an elongation of 300%. The filament 102 of not less than 17 dtex and not more than 45 dtex elongated to 300% has an outer diameter of not more than 0.04 mm. Therefore, use of such a filament 102 does not hinder the size reduction of the endoscope woven cable 100.

After weaving the filament 102 through the plural cables 101, the cables 101 are gathered together by a force of the filament 102 to recover from elongation. However, an excessive stress is not applied to the plural cables 101 by the filament 102 which has a high rate of elongation. Therefore, even if the outer diameter of the cable 101 is small, stress capable of bending the cable 101 with a small bend radius is not applied to the cables 101 by the filament 102 and it is thus possible to prevent the cables 101 from undulating or being broken due to the undulating.

As a result, it is possible to reduce distances (wiring pitch) between the plural cables 101 without applying an excess load to the plural cables 101 and thereby to reduce the width of the endoscope woven cable 100.

In addition, the filament 102 can be still sufficiently elongated even after being woven through the plural cables 101, and thus provides the widthwise stretching/contracting function of the endoscope woven cable 100.

Since this function allows the endoscope woven cable 100 to sufficiently follow bends or twists of the endoscope cable, it is possible to improve flexibility and twist resistance of the endoscope cable.

The rigidity-imparting wires 103 are preferably arranged at both ends and in the middle of the plural cables 101 in the alignment direction thereof.

The reason why the rigidity-imparting wires 103 are arranged at the both ends of the plural cables 101 in the alignment direction is as follows: when the filament 102 is woven through the plural cables 101, the both ends of the plural cables 101 in the alignment direction receive the strongest stress. Therefore, the rigidity-imparting wires 103, which are virtually not broken, need to be arranged at the both ends of the plural cables 101 in the alignment direction to prevent breakage of the other cables 101 (optical fiber cable(s), coaxial wire(s) and insulated wire(s), etc.).

Meanwhile, the reason why the rigidity-imparting wire 103 is arranged in the middle of the plural cables 101 in the alignment direction is as follows: stress is applied to the middle cable 101 when weaving the filament 102 to pass over/under one cable 101 as one unit at the middle of the endoscope woven cable 100 in the width direction as described above, and such a middle cable 101 needs to be the rigidity-imparting wire 103 to prevent breakage of the other cables 101.

Furthermore, the rigidity-imparting wires 103 are preferably symmetrically positioned about the center of the plural cables 101 in the alignment direction.

This configuration allows stress applied to the endoscope woven cable 100 to be uniformly dispersed throughout the entire endoscope woven cable 100.

The outer diameter of the rigidity-imparting wire 103 is preferably equal to or smaller than that of the rest of the cables 101.

The reason why the outer diameter of the rigidity-imparting wire 103 is equal to or smaller than that of the rest of the cables 101 is as follows: use of the rigidity-imparting wire 103 having a greater outer diameter than the other cables 101 makes difficult to sufficiently bind and fix the plural cables 101 by the filament 102 and also causes an excessive increase in rigidity of the endoscope woven cable 100 and a resulting decrease in capability of the endoscope woven cable 100.

Particularly when the outer diameter of the rigidity-imparting wire 103 is smaller than that of the other cables 101, it is possible to fold the endoscope woven cable 100 at the positions of the rigidity-imparting wires 103 and it is also easy to maintain the folded shape. In other words, it is possible to design a suitable folded shape according to wiring layout by selecting arrangement positions of the rigidity-imparting wires 103.

Considering that the outer diameter of the other cables 101 is about not less than 0.18 mm and not more than 0.25 mm, the outer diameter of the rigidity-imparting wire 103 is preferably about not less than 0.03 mm and not more than 0.15 mm. Furthermore, the rigidity-imparting wire 103 is preferably a stainless steel wire or a steel wire, etc., which has electrical conductivity in addition to excellent rigidity.

As a result, in case that a shielding tape is wound around the endoscope woven cable 100, measures against noise can be taken easily at the time of terminating the cable by reducing the potential of the rigidity-imparting wires 103 to ground potential since the rigidity-imparting wires 103 are in contact with the shielding tape. Meanwhile, since the shielding tape is not more than 0.1 mm in thickness, the size of the endoscope woven cable 100 hardly changes even if the shielding tape is wound around the endoscope woven cable 100.

In the endoscope woven cable 100 described above in which the plural cables 101 include at least one rigidity-imparting wire 103, even if rigidity of the endoscope woven cable 100 is reduced due to an increase in flexibility caused by reducing the diameter of the cables 101, the reduced rigidity is covered by the rigidity-imparting wires 103 and this allows the cables 101 have a reduced diameter while suppressing a decrease in rigidity.

In addition, in the endoscope woven cable 100, since the majority of the load generated by bends or twists can be transferred to the filament 102 with excellent stretch properties, it is possible to withstand the load generated by bends or twists and thus possible to prevent breakage of the cables 101 caused by the bends or the twists.

Furthermore, in the endoscope woven cable 100, the plural cables 101 are less likely to separate even when subjected to bends or twists since the plural cables 101 are integrated by the filament 102. Therefore, the cables 101 do not protrude from the endoscope woven cable 100 and thus do not receive an excessive load.

In addition, from the viewpoint of termination, the endoscope woven cable 100 is advantageous in that it is easy to sort out the cables 101 at the time of terminating the cable since the plural cables 101 are unified by the filament 102 and the arrangement thereof is consistent along the longitudinal direction.

Next, an endoscope cable of the invention will be described.

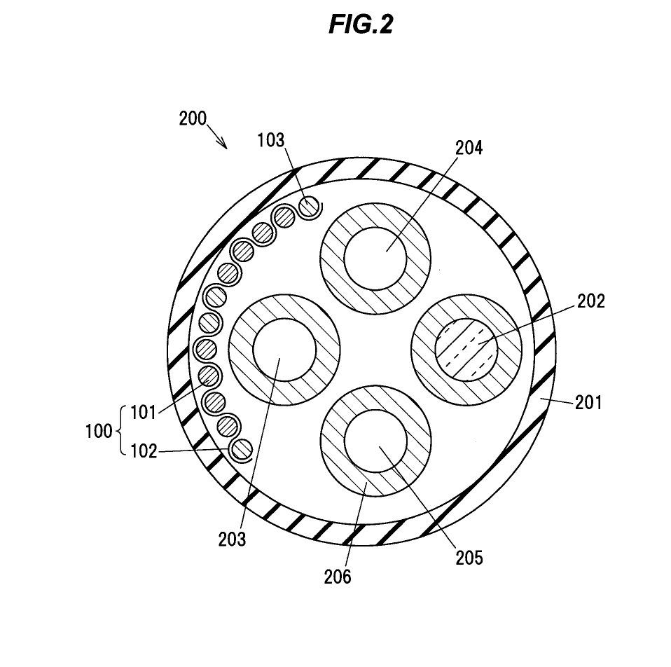

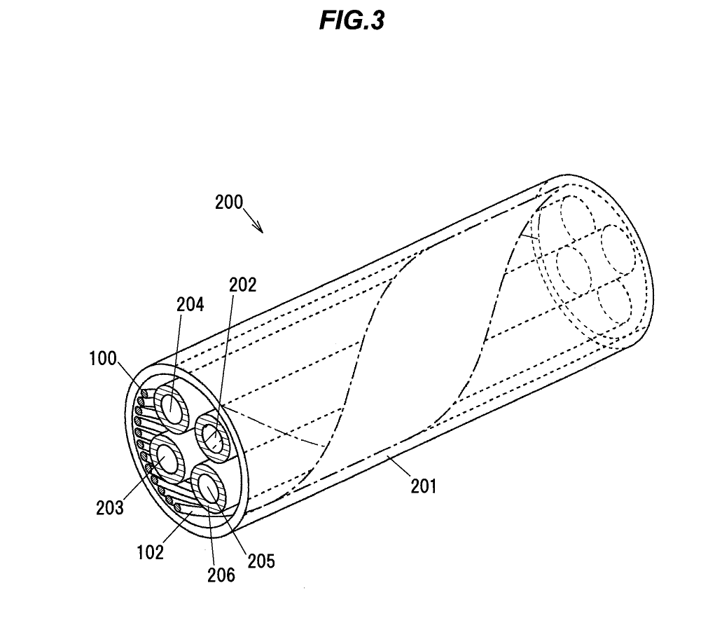

As shown in FIGS. 2 and 3, an endoscope cable 200 in the present embodiment is provided with a flexible hollow cylindrical endoscopic tube 201 and the endoscope woven cable 100 arranged inside the endoscopic tube 201.

Although the endoscope woven cable 100 as a medical endoscope woven cable used for a medical endoscopic device with solid-state image sensor has been described as an example in the present embodiment, the basic structure is also similar in the endoscope woven cable 100 as an industrial endoscope woven cable used for an industrial endoscopic device with solid-state image sensor.

In addition to the endoscope woven cable 100, the endoscope cable 200 also has a light guide 202, an air insufflation/water delivery channel 203, a suction channel 204 and a treatment tool channel 205, etc., which are arranged inside the endoscopic tube 201 and are used for in vivo observation or treatment. The light guide 202, the air insufflation/water delivery channel 203, the suction channel 204 and the treatment tool channel 205, etc., are each protected by a protective tube 206.

Preferably, the endoscope woven cable 100 is spirally arranged along the inner surface of the endoscopic tube 201 or is spirally wound around the protective tubes 206. This configuration prevents tangling of the endoscope woven cable 100 with the light guide 202, the air insufflation/water delivery channel 203, the suction channel 204 and the treatment tool channel 205, etc., thereby preventing breakage of the endoscope woven cable 100.

In the endoscope cable 200 using the endoscope woven cable 100 in which the plural cables 101 include at least one rigidity-imparting wire 103, even if rigidity of the endoscope woven cable 100 is reduced due to an increase in flexibility caused by reducing the diameter of the cables 101, the reduced rigidity is covered by the rigidity-imparting wires 103, this allows the cables 101 have a reduced diameter while suppressing a decrease in rigidity, and the endoscope woven cable 100 thus can be easily inserted into the endoscopic tube 201 and arranged at an appropriate position.

Therefore, it is possible to further reduce the diameter of the endoscope cable 200 as compared to conventional endoscope cables and thus to meet the demand of the market.

It should be noted that the invention is not intended to be limited to the embodiment, and the various kinds of modifications can be implemented without departing from the gist of the invention.

For example, although the endoscope woven cable 100 described in the present embodiment electrically and/or optically connects an endoscopic device main body to a solid-state image sensor and also supplies electric power from the endoscopic device main body to the solid-state image sensor and transmits electrical and/or optical signals between the endoscopic device main body and the solid-state image sensor, the endoscope woven cable 100 may be used in an ultrasonic endoscope device to electrically and/or optically connect an endoscopic device main to an ultrasound probe and also to supply electric power from the endoscopic device main body to the ultrasound probe and to transmit electrical and/or optical signals between the endoscopic device main body and the ultrasound probe.

What is claimed is:

1. An endoscope woven cable, comprising:
a plurality of cables arranged in parallel; and
a filament woven through the plurality of cables in an alignment direction thereof,
wherein the plurality of cables comprise at least one rigidity-imparting wire.

2. The endoscope woven cable according to claim 1, wherein the rigidity-imparting wire is arranged at both ends and in a middle of the plurality of cables in the alignment direction thereof.

3. The endoscope woven cable according to claim 1, wherein the rigidity-imparting wire is symmetrically positioned about a center of the plurality of cables in the alignment direction thereof.

4. The endoscope woven cable according to claim 1, wherein an outer diameter of the rigidity-imparting wire is not greater than that of a rest of the plurality of cables.

5. The endoscope woven cable according to claim 1, wherein the rigidity-imparting wire comprises a bare stainless steel wire or a bare steel wire.

6. An endoscope cable, comprising the endoscope woven cable according to claim 1, wherein the endoscope woven cable is spirally arranged inside an endoscopic tube.
